(19) United States
(12) Patent Application Publication
(10) Pub. No.: US 2023/0263664 A1
(43) Pub. Date: Aug. 24, 2023

(54) TREATMENT APPARATUS FOR AN EYE TREATMENT, METHOD, COMPUTER PROGRAM AND COMPUTER-READABLE MEDIUM

(71) Applicant: SCHWIND EYE-TECH-SOLUTIONS GMBH, Kleinostheim (DE)

(72) Inventors: Samuel ARBA MOSQUERA, Aschaffenburg (DE); Shwetabh VERMA, Aschaffenburg (DE)

(21) Appl. No.: 18/168,923

(22) Filed: Feb. 14, 2023

(30) Foreign Application Priority Data
Feb. 18, 2022 (DE) 10 2022 103 899.8

(51) Int. Cl. A61F 9/008

(52) U.S. Cl. CPC A61F 9/00814

(57) ABSTRACT

The invention relates to a treatment apparatus for an eye treatment, at least comprising: at least one laser beam source configured for emission of laser pulses, at least one beam exit device, which is configured to pass the respective laser pulses with a predetermined laser pulse cross-sectional profile to respective impingement positions in a treatment surface of a preset treatment volume of an eye to be treated, a control device, which is configured to ascertain coordinates of the respective impingement positions in the treatment surface according to at least two predetermined coordinate ascertaining methods for the preset treatment volume and the predetermined laser pulse cross-sectional profile. The control device is configured to ascertain respective expectable treatment roughnesses of the treatment volume depending on coordinate ascertaining method, according to a predetermined roughness ascertaining method for the at least two coordinate ascertaining methods, to determine the predetermined coordinate ascertaining method of a smallest expectable treatment roughness depending on coordinate ascertaining method as the coordinate ascertaining method to be adjusted, and to adjust the coordinates of the respective impingement positions according to the coordinate ascertaining method to be adjusted, in the at least one beam exit device.

### FIELD

The present invention relates to a treatment apparatus for an eye treatment. In addition, the invention relates to a method for controlling the treatment apparatus, to a computer and to a non-transitory computer-readable medium.

### BACKGROUND

Treatment apparatuses and methods for controlling ophthalmologic lasers for correcting an optical visual disorder and/or pathologically or unnaturally altered areas of the cornea are known in the prior art. Therein, a pulsed laser and a beam focusing device can for example be formed such that laser pulses effect a photodisruption and/or photoablation in a focus located within the organic tissue to remove a tissue, in particular a tissue lenticule, from the cornea.

In performing ablation methods, it is desired that a surface roughness of surfaces of a volume ablated from the tissue is minimum. The surface roughness of a treatment volume ablated by means of laser pulses is influenced by multiple parameters of the laser treatment. For example, the surface roughness depends on a laser pulse cross-sectional profile of the laser pulses, which are used for ablating the treatment volume. The laser pulse cross-sectional profile describes a radial intensity distribution of the radiant exposure of the laser pulses starting from a center. Furthermore, the surface roughness depends on a pattern, which the coordinates of the impingement points of the laser pulses form in an ablation surface of the ablation volume.

CN 1528256 A discloses a method and an apparatus for laser pulse ablation of corneal tissue with layer scanning. In the method, laser pulses are generated by the apparatus, wherein the corneal tissue is scanned with the laser pulses with high speed in two directions, to reach certain points on the corneal tissue within an adjusted area of action according to an algorithm adjusted by a program.

In the publication Verma, S., Hesser, J., & Arba-Mosquera, S. (2017). Optimum laser beam characteristics for achieving smoother ablations in laser vision correction. Investigative ophthalmology & visual science, 58(4), 2021-2037, a simulation model for the simulation of a shot-by-shot ablation method was designed. The effects of laser beam characteristics like super Gaussian order, cut-off radius, spot geometry, spot overlap and grid geometry were tested for their influence on a smoothness of the ablation.

The known methods allow a reduction of the surface roughness of surfaces for certain treatment approaches. However, the surface roughness of the surfaces of ablated treatment volumes further increases with an increasing ablation depth.

### SUMMARY

The invention is based on the object to provide a treatment apparatus, which allows an ablation of treatment volumes with a reduced surface roughness.

This object is solved by the treatment apparatus according to the invention according to the features of claim 1, the method according to the invention according to the features of claim 12, the computer program according to the invention according to the features of claim 14 and the computer-readable medium according to the invention according to the features of claim 15. Advantageous configurations with convenient developments of the invention are specified in the respective dependent claims, wherein advantageous configurations of each inventive aspect are to be regarded as advantageous configurations of the respectively other inventive aspects.

A first aspect of the invention relates to a treatment apparatus for an eye treatment. It is provided that the treatment apparatus comprises at least one laser beam source, which is configured for emission of laser pulses. The treatment apparatus comprises at least one beam exit device, which is configured to pass the respective laser pulses with a predetermined laser pulse cross-sectional profile to respective impingement positions in a treatment surface of a preset treatment volume of an eye to be treated.

The treatment apparatus comprises a control device, which is configured to ascertain the coordinates of the respective impingement positions of the laser pulses in the treatment surface according to at least two predetermined coordinate ascertaining methods for the preset treatment volume and the predetermined laser pulse cross-sectional profile. In other words, the control device is configured to provide the coordinates, to which the respective laser pulses are guided in the treatment surface of the preset treatment volume to ablate the preset treatment volume. The control device is configured to perform the coordinates according to the at least two coordinate ascertaining methods depending on the preset laser pulse cross-sectional profile. The coordinate ascertaining methods are designed such that they instruct the control device for providing the coordinates of the respective impingement positions of the laser pulses in the treatment surface for the preset laser pulse cross-sectional profile. The predetermined coordinate ascertaining methods can for example describe respective patterns and/or algorithms, which preset a setting of the coordinates for the treatment volume. The coordinates, which are ascertained by the respective coordinate ascertaining methods, can differ from each other such that at least two respective coordinate sets of the coordinates can be provided by the at least two coordinate ascertaining methods.

The control device is configured to ascertain respective treatment roughnesses of the treatment volume depending on coordinate ascertaining method expectable for the preset treatment volume, according to a predetermined roughness ascertaining method for the at least two coordinate ascertaining methods. In other words, it is provided that the control device is configured to ascertain for the at least two coordinate sets ascertained by the respective coordinate ascertaining methods, according to the predetermined roughness ascertaining method, which treatment roughness the treatment volume has after the laser pulses have been guided to the coordinates of the respective impingement positions to ablate the treatment volume. For example, the control device can be configured to perform simulations as the predetermined roughness ascertaining method, which simulate the ablation of the treatment volume and an arising surface of the treatment volume. The treatment roughness can for example be specified as a mean roughness value, square roughness or averaged roughness depth of the surfaces, which the treatment volume comprises.

The control device is configured to determine the coordinate ascertaining method of a smallest expectable treatment roughness depending on coordinate ascertaining method as the coordinate ascertaining method to be adjusted, and to adjust the coordinates of the respective impingement positions according to the coordinate ascertaining method to be adjusted, in the at least one beam exit device. In other words, the control device is configured to compare the expectable treatment roughnesses depending on coordinate ascertaining method, which arise for the coordinate sets of the respective coordinate ascertaining methods, to each other to ascertain, which one of the coordinate ascertaining methods results in coordinates, which result in a minimum treatment roughness of the treatment volume. The control device is configured to adjust the coordinates of the coordinate ascertaining method of the smallest expectable treatment roughness depending on coordinate ascertaining method in the at least one beam exit device before the eye treatment to guide the laser pulses to the associated coordinates.

The control device is configured to perform the roughness ascertaining method before performing the ablation method. In other words, it is provided that it is ascertained by the control device before performing the eye treatment, which one of the at least two coordinate ascertaining methods allows an ablation of the treatment volume with the lowest surface roughness. The control device is configured to adjust the coordinates ascertained according to the coordinate ascertaining method to be adjusted, in the beam exit device in order that the laser pulses are passed to the coordinates ascertained by means of the coordinate ascertaining method to be adjusted, in performing the eye treatment. By the invention, the advantage arises that the adequate coordinate ascertaining method to be adjusted is ascertained by means of a calculation or simulation before performing the eye treatment.

The invention also includes developments, by which additional advantages arise.

A development of the invention provides that the control device is configured to ascertain respective treatment roughnesses of the treatment volume depending on profile expectable for the preset treatment volume according to a predetermined profile ascertaining method for at least two potential laser pulse cross-sectional profiles of the laser pulse before the eye treatment. In other words, it is provided that it is ascertained by the control device according to the predetermined profile ascertaining method, in which respective treatment roughness of the treatment volume depending on profile the at least two potential laser pulse cross-sectional profiles of the laser pulses result. The control device is configured to determine the laser pulse cross-sectional profile of a smallest expectable treatment roughness depending on profile as the predetermined laser pulse cross-sectional profile of the laser pulses and to adjust the predetermined laser pulse cross-sectional profile in the at least one beam exit device. In other words, it is provided to ascertain the laser pulse cross-sectional profile of the laser pulses, which results in a minimum treatment roughness of the treatment volume depending on profile. The treatment roughness depending on profile can for example be ascertained by simulations. By the development of the invention, the advantage arises that a portion of the surface roughness, which is attributable to the laser pulse cross-sectional profile, can be reduced by a choice of the most suitable laser pulse cross-sectional profile. The ascertainment of the preset laser pulse cross-sectional profile can be effected before the selection of the coordinate ascertaining method such that the suitable coordinate ascertaining method can be effected for the preset laser pulse cross-sectional profile, for which a minimum treatment roughness of the treatment volume depending on profile is ascertained.

A development of the invention provides that the control device is configured to ascertain respective varied coordinate sets including varied coordinates of the respective impingement positions from the coordinates according to at least two predetermined jitter methods before the eye treatment, wherein the varied coordinates of the varied coordinate sets are shifted by respective jitter shifts with respect to the coordinates of the coordinate sets of the respective impingement positions in the treatment surface. The control device is configured to ascertain treatment roughnesses of the treatment volume depending on jitter expectable for the respective varied coordinate sets according to a predetermined jitter roughness ascertaining method, and to replace the coordinates with the varied coordinates of the varied coordinate set of a smallest expectable treatment roughness depending on jitter. In other words, it is provided that the control device is configured for performing the at least two jitter methods. The at least two jitter methods can preset respective jitter shifts of the coordinates of the respective impingement positions. The jitter shifts can be situated flatly in the treatment surface and include periodic and/or random portions. Thereby, the varied coordinates are shifted from the original coordinates of the impingement positions by the respective jitter shifts in the treatment surface. The control device is configured to evaluate the varied coordinate sets generated by the respective jitter methods, which include the varied coordinates, according to the predetermined jitter roughness ascertaining method, wherein respective treatment roughnesses depending on jitter are ascertained for the respective varied coordinate sets. The control device is configured to ascertain, which one of the varied coordinate sets results in a minimum treatment roughness of the treatment volume depending on jitter. The control device is configured to apply the jitter method, which results in the minimum treatment roughness depending on jitter, for performing the eye treatment method. At least one of the jitter methods can provide that jitter shifts are not effected. Thereby, the control device can ascertain if a variation of the coordinates influences the roughness, and refrain from a variation of the coordinates if the jitter method, which does not provide jitter shifts, results in the minimum treatment roughness of the treatment volume depending on jitter.

A development of the invention provides that the at least two predetermined coordinate ascertaining methods include at least one preset dithering method. The dithering method can for example preset a respective dithering matrix. The dithering matrix is also known as an index matrix, Bayer matrix or threshold map.

A development of the invention provides that the at least two predetermined coordinate ascertaining methods include at least one of the following predetermined dithering methods: Floyd-Steinberg, thresholding, random dithering, ordered dithering, Bayer matrix, minimized average error, Stucki dithering, Burkes dithering, Sierra dithering, two-row Sierra, filter lite, Atkinson dithering or gradient-based error-diffusion dithering. In other words, the control device is configured to apply at least one of the above mentioned dithering methods as the coordinate ascertaining method for ascertaining the coordinates of the respective impingement positions of the laser pulses in the treatment surface.

A development of the invention provides that the at least two potential laser pulse cross-sectional profiles include at least one Gaussian cross-sectional profile. In other words, at least one of the laser pulse cross-sectional profiles is a laser pulse cross-sectional profile, which describes a Gaussian distribution of an intensity of the laser pulse across the cross-section. In further other words, the control device is configured to control the beam exit device for generating a laser pulse, which has the Gaussian cross-sectional profile.

A development of the invention provides that the at least two potential laser pulse cross-sectional profiles include at least one pinhole cross-sectional profile. In other words, it is provided that at least one of the two laser pulse cross-sectional profiles is cut to a predetermined pinhole width. In other words, it is provided that a profile width is restricted to a pinhole width in at least one laser pulse cross-sectional profile. Therein, a course of the profile can have an intensity of a value of 0 from a certain distance. In further other words, the control device is configured to control the beam exit device for generating a laser pulse, which has a laser pulse cross-sectional profile, which is restricted to a pinhole width at least in one direction.

A development of the invention provides that the at least two potential laser pulse cross-sectional profiles have different full widths at half maximum. In other words, it is provided that the at least two laser pulse cross-sectional profiles differ from each other in their full widths at half maximum. In further other words, the control device is configured to control the beam exit device for outputting laser pulses, the laser pulse cross-sectional profiles of which differ from each other in their full width at half maximum.

A development of the invention provides that the at least two potential laser pulse cross-sectional profiles differ from each other in their maximum radiant exposures. In other words, it is provided that the at least two laser pulse cross-sectional profiles have different maximum radiant exposures. In further other words, it is provided that the radiant exposures of the at least two laser pulse cross-sectional profiles differ from each other in their maximum values.

A development of the invention provides that the at least two jitter methods include at least one random jitter method. In other words, at least one of the jitter methods is a random jitter method. The random jitter method can also be referred to as random jitter. The random jitter method is characterized in that the generated jitter shifts have a normal distribution. In further other words, the control device is configured to apply at least one random jitter method for generating the jitter shifts of the coordinates of the respective impingement positions of the laser pulses in the treatment surface.

A development of the invention provides that the at least two jitter methods include at least one deterministic jitter method. In other words, it is provided that at least one of the jitter methods is a deterministic jitter method for generating deterministic jitter shifts. The deterministic jitter method can for example generate jitter shifts, which include a periodic portion. In further other words, the control device is configured to apply at least one deterministic jitter method for generating the jitter shifts of the coordinates of the respective impingement positions of the laser pulses in the treatment surface.

A second aspect of the invention relates to a method for controlling a treatment apparatus by a control device. The control device is configured to perform the method for controlling a treatment apparatus. For example, the control device can be configured as a control chip, control appliance or application program (“app”). Preferably, the control device can comprise a processor device and/or a data storage. By a processor device, an appliance or an appliance component for electronic data processing is understood. For example, the processor device can comprise at least one microcontroller and/or at least one microprocessor. Preferably, a computer program for performing the method according to the invention can be stored on the optional data storage and/or a computer-readable medium. The computer program can then be configured, upon execution by the processor device, to cause the control device to perform one of the above-described embodiments of one or both methods according to the invention.

The control device is configured for controlling the treatment apparatus. The method includes at least the following steps.

In a step, ascertaining coordinates of respective impingement positions in a treatment surface is effected for a preset treatment volume and a predetermined laser pulse cross-sectional profile according to a predetermined coordinate ascertaining method before performing the treatment. In other words, it is provided that the coordinates are ascertained by the control device, which have the respective impingement positions of the laser pulse in the treatment surface for the preset treatment volume. The ascertainment of the coordinates is effected considering the predetermined laser pulse cross-sectional profile, which the laser pulses have. The ascertainment of the coordinates of the respective impingement positions is effected according to the predetermined coordinate ascertaining method, which for example presets a dithering matrix, to determine the coordinates of the impingement positions of the laser pulses in the treatment surface.

In a further step, ascertaining respective treatment roughnesses of the treatment volume depending on coordinate ascertaining method expectable for the preset treatment volume is effected according to a predetermined roughness ascertaining method for the at least two coordinate ascertaining methods.

Subsequently, determining the coordinate ascertaining method of a smallest expectable treatment roughness depending on coordinate ascertaining method as the predetermined coordinate ascertaining method is effected. In other words, that coordinate ascertaining method is ascertained, which generates coordinates, which result in the smallest expectable treatment roughness depending on coordinate ascertaining method.

In a further step, a control of the beam exit device by the control device is effected. In other words, it is provided that the beam exit device is controlled by the control device, whereby the laser pulses are guided to the respective impingement positions in the treatment surface. The adjustment of the coordinates in the beam exit device can be sequentially effected such that the beam exit device can be sequentially adjusted after emitting a respective one of the pulses to the respective impingement positions.

Further features and the advantages thereof can be taken from the descriptions of the first inventive aspect, wherein advantageous configurations of each inventive aspect are to be regarded as advantageous configurations of the respectively other inventive aspect.

A development of the invention provides that the method includes at least the following steps. Ascertaining the respective treatment roughness of the treatment volume depending on profile expectable for the preset treatment volume for the at least two potential laser pulse cross-sectional profiles according to the predetermined profile ascertaining method by the control device. In other words, it is provided that it is ascertained by the control device before performing the treatment method, in which treatment roughness depending on profile the use of the respective at least two laser pulse cross-sectional profiles of the laser pulses results. In a further step, it is provided that the laser pulse cross-sectional profile is ascertained by the control device, for which the smallest expectable treatment roughness depending on profile results. This laser pulse cross-sectional profile is adjusted as the predetermined laser pulse cross-sectional profile for the treatment method. In other words, the laser pulse cross-sectional profile, which results in the minimum treatment roughness depending on profile is ascertained for performing the treatment and adjusted in the beam exit device.

A development of the invention provides that the method includes at least the following steps. Ascertaining respective varied coordinate sets including varied coordinates of the coordinates according to at least two jitter methods by the control device, wherein the varied coordinates of the varied coordinate sets are shifted by respective jitter shifts in the treatment surface with respect to the coordinates in the treatment surface. In other words, it is provided that the coordinates of the coordinate set are varied according to the at least two jitter methods to generate the varied coordinates of the varied coordinate sets before performing the treatment method. Ascertaining the respective treatment roughnesses of the treatment volume depending on jitter expectable for the preset treatment volume is effected for the coordinate sets according to the predetermined jitter roughness ascertaining method. In other words, the treatment roughnesses depending on jitter are ascertained for the respective varied coordinate sets. Thereby, it can be ascertained, in which treatment roughness depending on jitter an application of the respective jitter methods would result. In a further step, it is provided that the coordinates are replaced with the varied coordinates of the coordinate set of the smallest expectable treatment roughness depending on jitter.

A third aspect of the invention relates to a computer program including commands, which cause a control device to execute the method steps according to the second inventive aspect.

Further features and the advantages thereof can be taken from the descriptions of the first and the second inventive aspect, wherein advantageous configurations of each inventive aspect are to be regarded as advantageous configurations of the respectively other inventive aspect.

A fourth aspect of the invention relates to a computer-readable medium, on which the computer program according to the third inventive aspect is stored.

Further features and the advantages thereof can be taken from the descriptions of the first, the second and the third inventive aspect, wherein advantageous configurations of each inventive aspect are to be regarded as advantageous configurations of the respectively other inventive aspect.

### BRIEF DESCRIPTION OF THE DRAWINGS

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 is a schematic representation of a treatment apparatus for an eye treatment.

FIG. 2 is a schematic representation of two patterns.

FIG. 3 is a schematic representation of a shift of the coordinates by jitter shifts.

FIG. 4 is a schematic representation of possible laser pulse cross-sectional profiles.

FIG. 5 is a schematic representation of a procedure of a method for controlling the treatment apparatus.

### DETAILED DESCRIPTION

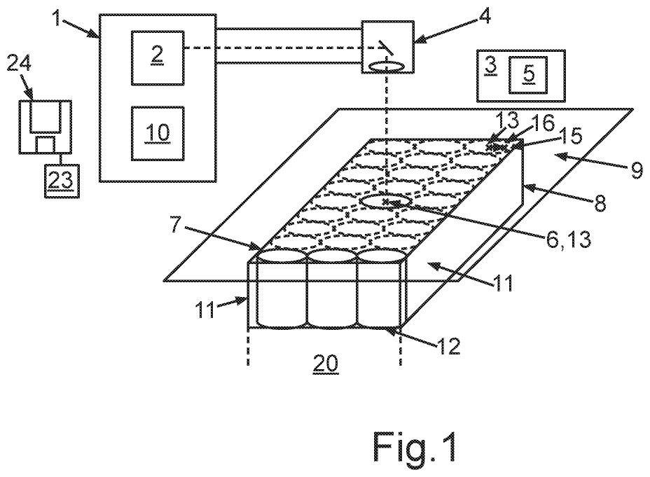

FIG. 1 shows a schematic representation of a treatment apparatus for an eye treatment. The treatment apparatus 1 comprises at least one laser beam source 2, which may be configured to emit laser pulses 3. In addition, the treatment apparatus 1 may comprise at least one beam exit device 4, which may be configured to guide the respective laser pulses 3 with a predetermined laser pulse cross-sectional profile 5 to respective impingement positions 6 onto a treatment surface 7 of a preset treatment volume 8 of an eye 9 to be treated. The treatment apparatus 1 may comprise a control device 10, which may be configured to control the beam exit device 4 and the laser beam source 2 for ablating the treatment volume 8. It may be provided that the ablation of the treatment volume 8 is preferably effected such that lateral surfaces 11 of the treatment volume 8 and a basic surface 12 of the treatment volume 8 have a minimum surface roughness. The surface roughness of the lateral surfaces 11 of the treatment volume 8 and the surface roughness of the basic surface 12 of the treatment volume 8 may be combined as treatment roughness of the treatment volume 8. The treatment roughness of the treatment volume 8 may depend on multiple factors. In order to be able to overall minimize the treatment roughness of the treatment volume 8, it may therefore be required to individually optimize the individual parameters, which influence the surface roughness.

The surface roughness may depend on the laser pulse cross-sectional profile 5 of the laser pulses 3. By the choice of the predetermined laser pulse cross-sectional profile 5, it is thus possible to reduce the treatment roughness. The control device 10 may be configured to ascertain respective treatment roughnesses of the treatment volume 8 depending on profile expectable for the preset treatment volume 8, according to a predetermined profile ascertaining method for at least two potential laser pulse cross-sectional profiles 5 of the laser pulses 3 before the eye treatment. In other words, at least two laser pulse cross-sectional profiles 5 may be stored in the control device 10, which may have the laser pulses 3 generated by the treatment apparatus 1. The laser pulse cross-sectional profiles 5 may for example include a Gaussian profile. The laser pulse cross-sectional profiles 5 may describe an intensity distribution of the respective laser pulses 3 across a radius of the laser pulses 3. Depending on the laser pulse cross-sectional profiles 5 of the respective laser pulses 3, respective treatment roughnesses of the treatment volume 8 may arise. The control device 10 may be configured to ascertain respective treatment roughnesses of the treatment volume 8 depending on profile expectable for the preset treatment volume 8, according to the predetermined profile ascertaining method for the at least two potential laser pulse cross-sectional profiles 5 of the laser pulses 3. In other words, the control device 10 may be configured to ascertain the respective treatment roughnesses of the treatment volume 8 depending on profile according to the predetermined profile ascertaining method for the at least two potential laser pulse cross-sectional profiles 5, which would arise upon choice of the respective laser pulse cross-sectional profile 5. The control device 10 may be configured to ascertain the laser pulse cross-sectional profile 5 of the smallest expectable treatment roughness depending on profile as the predetermined laser pulse cross-sectional profile 5, and to adjust the predetermined laser pulse cross-sectional profile 5 in the at least one beam exit device 4 during the treatment method.

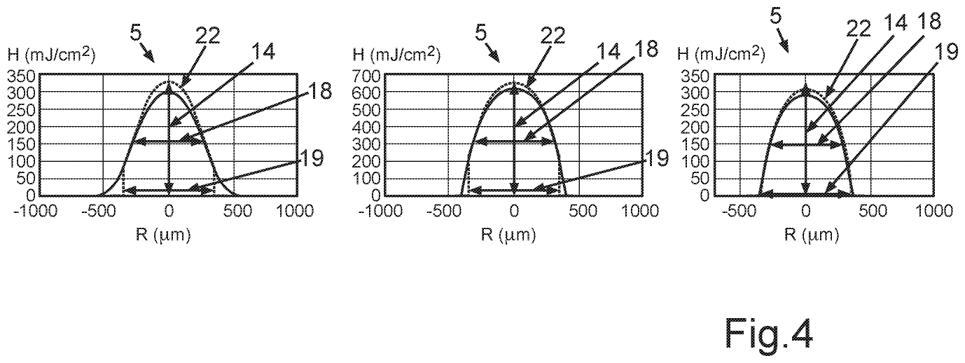

Possible ones of the laser pulse cross-sectional profiles 5 are shown in FIG. 4. They may differ from each other in their maximum radiant exposures 14, their courses and/or their full widths at half maximum 18. It may be provided that the laser pulse cross-sectional profiles 5 may be laterally restricted by a shutter. Thereby, edge areas, which are outside of a selected distance to the center, may be removed. Accordingly, the laser pulse cross-sectional profiles 5 may be restricted to a pinhole width 19.

The control device 10 may be configured to ascertain the coordinates 13 of the respective impingement positions 6 of the laser pulses 3 in the treatment surface 7 according to at least two predetermined coordinate ascertaining methods for the preset treatment volume 8 and the predetermined laser pulse cross-sectional profile 5. The at least two coordinate ascertaining methods may include matrices, which may allow ascertaining the coordinates 13 of the respective impingement positions 6 in the treatment surface 7. The coordinate ascertaining methods may for example include the following methods: Floyd-Steinberg, thresholding, random dithering, ordered dithering, Bayer matrix, minimized average error, Stucki dithering, Burkes dithering, Sierra dithering, two-row Sierra, filter lite, Atkinson dithering or gradient-based error-diffusion dithering.

By the respective coordinate ascertaining methods, different coordinate sets of the respective coordinates 13 of the impingement positions 6 in the treatment surface 7 may be ascertained. The treatment roughnesses depending on coordinate ascertaining method, which result therefrom for the treatment volume 8, may differ from each other. Thus, it may be required to minimize the treatment roughness influenced by the choice of the coordinate ascertaining method.

It may be provided that the control device 10 is configured to ascertain the treatment roughnesses of the treatment volume 8 depending on coordinate ascertaining method expectable for the at least two coordinate ascertaining methods for the preset treatment volume 8 according to a predetermined dithering roughness ascertaining method before performing the treatment method. In other words, the control device 10 is configured to ascertain, which treatment roughnesses depending on coordinate ascertaining method would arise for the respective coordinate ascertaining methods in performing the treatment. The control device 10 may be configured to compare the treatment roughnesses depending on coordinate ascertaining method of the respective coordinate ascertaining methods to each other and to determine the coordinate ascertaining method of a smallest expectable treatment roughness depending on coordinate ascertaining method as the predetermined coordinate ascertaining method. This means that the coordinates 13 of the respective impingement positions 6 are adjusted according to the predetermined coordinate ascertaining method in the at least one beam exit device 4 by the control device 10 during the treatment method.

A possibility of influencing the treatment roughness is in an adaptation of the coordinates 13 of the impingement positions 6 to varied coordinates 15 of the impingement positions 6 by means of so-called jitter methods. Jitter methods may generate jitter shifts 16 from the coordinates 13 to the varied coordinates 15, which may be effected in random manner according to so-called random jitter methods or in deterministic manner according to so-called deterministic jitter methods. The control device 10 may be configured to generate respective varied coordinate sets including the varied coordinates 15 of the respective impingement positions 6 from the coordinates 13 according to at least two jitter methods before the eye treatment, wherein the varied coordinates 15 are shifted by respective jitter shifts 16 with respect to the coordinates 13 of the treatment surface 7. The control device 10 may be configured to ascertain respective expectable treatment roughnesses of the treatment volume 8 depending on jitter for the respective coordinate sets according to the predetermined jitter roughness ascertaining method, and to replace the coordinates 13 with the varied coordinates 15 of the coordinate set of a smallest expectable treatment roughness depending on jitter. In other words, the control device 10 may be configured to ascertain the treatment roughnesses depending on jitter for the respective jitter methods, which would arise for the respective jitter methods. The jitter method, which would result in a smallest expectable treatment roughness depending on jitter is used to replace the coordinates 13 with the varied coordinates 15.

It may be provided that the ablation of the preset treatment volume 8 may be effected in layers, wherein layers 17 of the treatment volume 8 arranged one below the other may differ from each other in their predetermined laser pulse cross-sectional profile 5, the selected coordinate ascertaining method and/or the selected jitter method. Thereby, surface roughnesses in the basic surface 12 of the treatment volume 8 may for example be reduced. Similarly, it is possible to separately consider effects depending on depth in different depths of the treatment volume 8.

A computer program 23 may include commands, which cause the treatment apparatus 1 to perform a method for the eye treatment, for example controlled by the control device 10. The computer program 23 may be stored on a computer-readable medium, for example a hard disk, an USB stick or a CD.

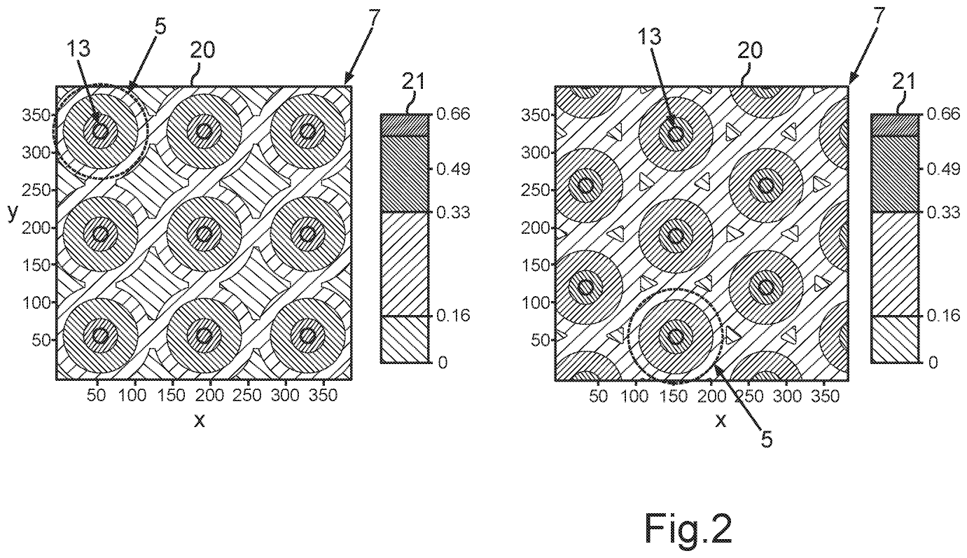

FIG. 2 shows a schematic representation of two patterns. The two patterns 20 show respective distributions of cutting depths 21 in μm in the treatment surface 7, which may arise in an ablation of the treatment volume 8. The treatment surface 7 may be situated in an X-Y plane. The patterns 20 may arise due to the positions 13, which are ascertained generated according to respective ones of the dithering methods. The positions 13 may be ascertained by two different ones of the coordinate ascertaining methods for the identical laser pulse cross-sectional profile 5. Depending on the selected coordinate ascertaining method, the surface roughnesses of the lateral surfaces 11 and/or the basic surface 12 of the treatment volume 8 may differ from each other.

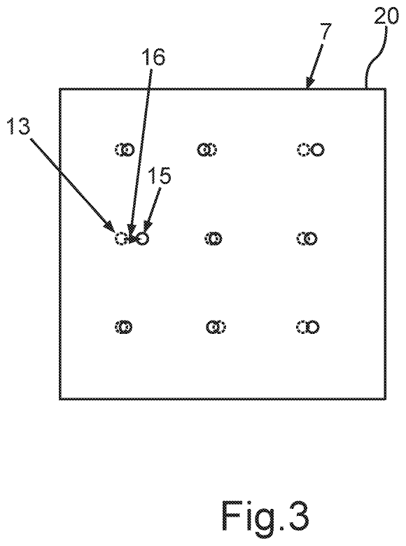

FIG. 3 shows a schematic representation of a shift of coordinates by jitter shifts The respective jitter method may preset, by which jitter shifts 16 the varied coordinates 15 of the impingement points 6 are shifted with respect to the coordinates 13 of the impingement points 6. The jitter method may for example be a random jitter method, which may preset a shift of the coordinates 13 to the varied coordinates 15 by random values. A deterministic jitter method may also be preset, which may for example preset periodic jitter shifts 16 across adjacent impingement points 6.

FIG. 4 shows possible laser pulse cross-sectional profiles. The radial course 22 of the radiant exposure H (radiant exposure) across the radial cross-section R of the laser cross-sectional profiles is illustrated. The laser pulse cross-sectional profiles 5 may differ from each other in their courses 22, their maximum radiant exposures 14 and/or their full widths at half maximum 18. It may be provided that laser pulse cross-sectional profiles 5 are guided through a shutter to limit them to a pinhole width 19.

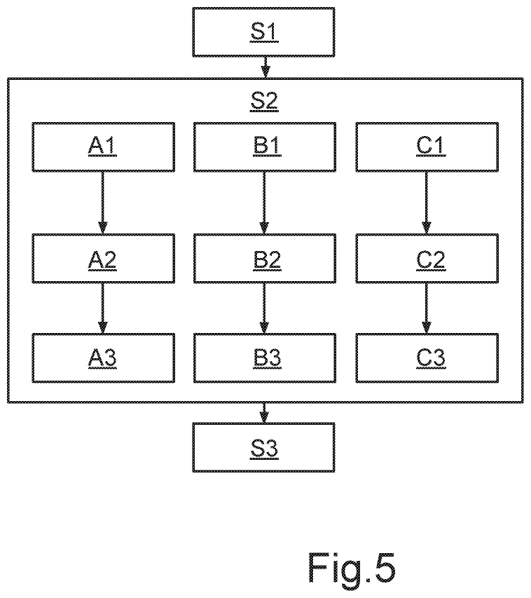

FIG. 5 shows a schematic representation of a procedure of a method for controlling the treatment apparatus.

In a first step S1, a treatment volume 8 to be ablated, which includes a treatment surface 7 of an eye 9, may be preset to the control device 10. In other words, the control device 10 may receive a treatment volume 8 to be treated by the treatment apparatus 1.

It may be provided that parameters for performing the treatment method are ascertained by the control device 10 in a second step S2 of the method. The ascertainment may include multiple partial steps, which may be performed in parallel or sequential manner.

In the second step S2, a coordinate ascertaining method and/or a jitter method and/or a laser pulse cross-sectional profile 5 may be ascertained by the control device 10, which may result in a minimum treatment roughness of the treatment volume 8. The ascertainment of the preset coordinate ascertaining method, the ascertainment of the preset laser pulse cross-sectional profile 5 and/or the ascertainment of the preset jitter method may be effected in parallel or sequential manner.

In a partial step A1 of the step S2, at least two of the laser pulse cross-sectional profiles 5, which may have the laser pulses 3 output by the treatment apparatus 1, may be retrieved from a storage by the control device 10.

In a partial step A2 of the step S2, respective treatment roughnesses of the treatment volume 8 depending on profile expectable for the preset treatment volume 8 may be ascertained according to a predetermined profile ascertaining method for the at least two laser pulse cross-sectional profiles 5 of the laser pulses 3 by the control device 10.

In a partial step A3 of the step S2, that one of the at least two laser pulse cross-sectional profiles 5 may be ascertained as the predetermined laser pulse cross-sectional profile 5 by the control device 10, which has a smallest expectable treatment roughness depending on profile.

In a partial step B1 of the step S2, the control device 10 may ascertain an ascertainment of the coordinates 13 of the respective impingement positions 6 in the treatment surface 7 for the preset treatment volume 8 and the predetermined laser pulse cross-sectional profile 5 according to the respective coordinate ascertaining methods. In other words, the coordinates 13 of the respective impingement positions 6 are ascertained by the control device 10 according to at least two different coordinate ascertaining methods for the treatment surface 7.

In a partial step B2 of the step S2, respective treatment roughnesses of the treatment volume 8 depending on coordinate ascertaining method expectable for the preset treatment volume 8 may be ascertained according to a predetermined roughness ascertaining method for the at least two coordinate ascertaining methods. The treatment roughnesses depending on coordinate ascertaining method may for example be ascertained by means of simulations or mathematic methods.

In a partial step B3 of the step S2, the coordinate ascertaining method may be ascertained, which results in the smallest treatment roughness depending on coordinate ascertaining method. This coordinate ascertaining method may be used as the predetermined coordinate ascertaining method for performing the eye treatment by the control device 10.

In a partial step C1 of the step S2, it may be provided that the coordinates 13 of the impingement positions 6 are transformed to varied coordinates 15 according to at least two jitter methods by the control device 10, wherein the varied coordinates 15 may be shifted by jitter shifts 16 from the coordinates 13.

In a partial step C2 of the step S2, respective expectable treatment roughnesses of the treatment volume 8 depending on jitter may be ascertained according to a predetermined jitter roughness ascertaining method for the respective jitter methods by the control device 10.

In a partial step C3 of the step S2, it may be provided that the coordinates 13 are replaced with the varied coordinates 13 of the jitter method of a smallest expectable treatment roughness depending on jitter.

By ascertaining the coordinate ascertaining method, the jitter method and the laser pulse cross-sectional profile 5 before the treatment, which each result in a smallest treatment roughness of the treatment volume 8, a minimum treatment roughness of the treatment volume 8 may be achieved by the treatment.

The method steps may be repeated for different layers 17 of the treatment volume 8. The layers 17 may be partial volumes of the treatment volume 8, which may be arranged one below the other or next to each other with respect to a depth.

In a method step S3, the treatment method may be performed, wherein the treatment apparatus 1 may be controlled by the control device 10, in order that the treatment apparatus 1 outputs the laser pulses for treating the treatment volume 8.

By the invention, the advantage arises that the values, which result in a minimum roughness, are ascertained before performing the eye treatment.

### LIST OF REFERENCE CHARACTERS

1 Treatment apparatus
2 laser beam source
3 laser pulses
4 beam exit device
5 laser pulse cross-sectional profile
6 impingement positions
7 treatment surface
8 treatment volume
9 eye
10 control device
11 lateral surface
12 basic surface
13 coordinates
14 maximum radiant exposure
15 varied coordinates
16 jitter shift
17 layers
18 full width at half maximum
19 pinhole width
20 pattern
21 cutting depth
22 course of a laser pulse cross-sectional profile
23 computer program
24 computer-readable medium
S1-S3 method steps
A1-A3 partial method steps
B1-B3 partial method steps
C1-C3 partial method steps

1. A treatment apparatus for an eye treatment, at least comprising:
at least one laser beam source, configured for emission of laser pulses,
at least one beam exit device, which is configured to guide the respective laser pulses with a predetermined laser pulse cross-sectional profile to respective impingement positions in a treatment surface of a preset treatment volume of an eye to be treated,
a control device, which is configured to ascertain coordinates of the respective impingement positions in the treatment surface according to at least two predetermined coordinate ascertaining methods for the preset treatment volume and the predetermined laser pulse cross-sectional profile,
to ascertain respective treatment roughnesses of the treatment volume depending on coordinate ascertaining method expectable for the preset treatment volume, according to a predetermined roughness ascertaining method for the at least two predetermined coordinate ascertaining methods,
to determine the predetermined coordinate ascertaining method of a smallest expectable treatment roughness depending on coordinate ascertaining method as the coordinate ascertaining method to be adjusted, and
to adjust the coordinates of the respective impingement positions according to the coordinate ascertaining method to be adjusted, in the at least one beam exit device.

2. The treatment apparatus according to claim 1, wherein the control device is configured,
to ascertain respective treatment roughnesses of the treatment volume expectable for the preset treatment volume depending on profile, according to a predetermined profile ascertaining method for at least two potential laser pulse cross-sectional profiles of the laser pulses before the eye treatment,
to determine the potential laser pulse cross-sectional profile of a smallest expectable treatment roughness depending on profile as the predetermined laser pulse cross-sectional profile, and
to adjust the predetermined laser pulse cross-sectional profile in the at least one beam exit device.

3. The treatment apparatus according to claim 1, wherein the control device is configured
to ascertain respective coordinate sets including varied coordinates of the respective impingement positions from the coordinates according to at least two predetermined jitter methods before the eye treatment, wherein the varied coordinates of the coordinate sets are shifted by respective jitter shifts with respect to the coordinates in the treatment surface,
to ascertain respective expectable treatment roughnesses of the treatment volume depending on jitter according to a predetermined jitter roughness ascertaining method for the respective coordinate sets, and
to replace the coordinates with the varied coordinates of the coordinate set of a smallest expectable treatment roughness depending on jitter.

4. The treatment apparatus according to claim 1, wherein the at least two predetermined coordinate ascertaining methods include at least one preset dithering method.

5. The treatment apparatus according to claim 1, wherein the at least two predetermined coordinate ascertaining methods include at least one preset dithering method, and wherein the at least two predetermined coordinate ascertaining methods include at least one of the following predetermined dithering methods: Floyd-Steinberg, thresholding, random dithering, ordered dithering, Bayer matrix, minimized average error, Stucki dithering, Burkes dithering, Sierra dithering, two-row Sierra, filter lite, Atkinson dithering or gradient-based error-diffusion dithering.

6. The treatment apparatus according to claim 1, wherein the control device is configured,
to ascertain respective treatment roughnesses of the treatment volume expectable for the preset treatment volume depending on profile, according to a predetermined profile ascertaining method for at least two potential laser pulse cross-sectional profiles of the laser pulses before the eye treatment,
to determine the potential laser pulse cross-sectional profile of a smallest expectable treatment roughness depending on profile as the predetermined laser pulse cross-sectional profile, and
to adjust the predetermined laser pulse cross-sectional profile in the at least one beam exit device,
wherein the at least two potential laser pulse cross-sectional profiles include at least one Gaussian cross-sectional profile.

7. The treatment apparatus according to claim 1, wherein the control device is configured,
to ascertain respective treatment roughnesses of the treatment volume expectable for the preset treatment volume depending on profile, according to a predetermined profile ascertaining method for at least two potential laser pulse cross-sectional profiles of the laser pulses before the eye treatment,
to determine the potential laser pulse cross-sectional profile of a smallest expectable treatment roughness depending on profile as the predetermined laser pulse cross-sectional profile, and
to adjust the predetermined laser pulse cross-sectional profile in the at least one beam exit device,
wherein the at least two potential laser pulse cross-sectional profiles include at least one pinhole cross-sectional profile.

8. The treatment apparatus according to claim 1, wherein the control device is configured,
to ascertain respective treatment roughnesses of the treatment volume expectable for the preset treatment volume depending on profile, according to a predetermined profile ascertaining method for at least two potential laser pulse cross-sectional profiles of the laser pulses before the eye treatment,
to determine the potential laser pulse cross-sectional profile of a smallest expectable treatment roughness depending on profile as the predetermined laser pulse cross-sectional profile, and
to adjust the predetermined laser pulse cross-sectional profile in the at least one beam exit device,
wherein the at least two potential laser pulse cross-sectional profiles have different half widths at full maximum.

9. The treatment apparatus according to claim 1, wherein the control device is configured,
to ascertain respective treatment roughnesses of the treatment volume expectable for the preset treatment volume depending on profile, according to a predetermined profile ascertaining method for at least two potential laser pulse cross-sectional profiles of the laser pulses before the eye treatment,
to determine the potential laser pulse cross-sectional profile of a smallest expectable treatment roughness depending on profile as the predetermined laser pulse cross-sectional profile, and
to adjust the predetermined laser pulse cross-sectional profile in the at least one beam exit device,
wherein the at least two potential laser pulse cross-sectional profiles have various maximum radiant exposures.

10. The treatment apparatus according to claim 1, wherein the control device is configured
to ascertain respective coordinate sets including varied coordinates of the respective impingement positions from the coordinates according to at least two predetermined jitter methods before the eye treatment, wherein the varied coordinates of the coordinate sets are shifted by respective jitter shifts with respect to the coordinates in the treatment surface,
to ascertain respective expectable treatment roughnesses of the treatment volume depending on jitter according to a predetermined jitter roughness ascertaining method for the respective coordinate sets, and
to replace the coordinates with the varied coordinates of the coordinate set of a smallest expectable treatment roughness depending on jitter,
wherein the at least two predetermined jitter methods include at least one random jitter method.

11. The treatment apparatus according to claim 1, wherein the control device is configured
to ascertain respective coordinate sets including varied coordinates of the respective impingement positions from the coordinates according to at least two predetermined jitter methods before the eye treatment, wherein the varied coordinates of the coordinate sets are shifted by respective jitter shifts with respect to the coordinates in the treatment surface,
to ascertain respective expectable treatment roughnesses of the treatment volume depending on jitter according to a predetermined jitter roughness ascertaining method for the respective coordinate sets, and
to replace the coordinates with the varied coordinates of the coordinate set of a smallest expectable treatment roughness depending on jitter,
wherein the at least two predetermined jitter methods include at least one deterministic jitter method.

12. A method for controlling a treatment apparatus by a control device, which is configured for controlling the treatment apparatus, comprising at least the steps:
ascertaining coordinates of respective impingement positions in a treatment surface for a preset treatment volume and a predetermined laser pulse cross-sectional profile according to at least two predetermined coordinate ascertaining methods,
ascertaining respective treatment roughnesses of the treatment volume depending on coordinate ascertaining method expectable for the preset treatment volume, according to a predetermined roughness ascertaining method for the at least two predetermined coordinate ascertaining methods,
determining the predetermined coordinate ascertaining method of a smallest expectable treatment roughness depending on coordinate ascertaining method as a coordinate ascertaining method to be adjusted, and
adjusting the coordinates of the respective impingement positions of the coordinate ascertaining method to be adjusted, in the beam exit device.

13. The method for controlling the treatment apparatus according to claim 12, comprising the further steps:
ascertaining the respective treatment roughnesses of the treatment volume depending on profile expectable for the preset treatment volume for the at least two potential laser pulse cross-sectional profiles of the laser pulses according to the predetermined profile ascertaining method,
determining the laser pulse cross-sectional profile of the smallest expectable treatment roughness depending on profile as the predetermined laser pulse cross-sectional profile, and
adjusting the predetermined laser pulse cross-sectional profile in the at least one beam exit device,
and/or the further steps:
ascertaining respective coordinate sets including varied coordinates from the coordinates according to the at least two predetermined jitter methods, wherein the varied coordinates of the coordinate sets are shifted by respective jitter shifts in the treatment surface with respect to the coordinates in the treatment surface,
ascertaining the respective treatment roughnesses of the treatment volume depending on jitter expectable for the preset treatment volume for the respective coordinate sets according to the predetermined jitter roughness ascertaining method, and
replacing the coordinates with the varied coordinates of the coordinate set of the smallest expectable treatment roughness depending on jitter.

14. A computer program including commands, which cause a control device to execute the method steps according to claim 12.

15. A non-transitory computer-readable medium, on which the computer program according to claim 14 is stored.
